(19) United States
(12) Patent Application Publication
(10) Pub. No.: US 2023/0368923 A1
(43) Pub. Date: Nov. 16, 2023

(54) PERSONAL BIOMETRIC ASSESSMENT SYSTEM

(71) Applicant: LENOVO (SINGAPORE) PTE. LTD, Singapore (SG)

(72) Inventor: Marco M. Rengan, Chapel Hill, NC (US)

(21) Appl. No.: 17/742,492

(22) Filed: May 12, 2022

(51) Int. Cl. G16H 50/30; G16H 40/67

(52) U.S. Cl. CPC G16H 50/30; G16H 40/67

(57) ABSTRACT

A personal computer device includes a video camera, a microphone, and an infrared camera. The personal computer device receives face coloration video data of a user, breathing data of the user, and body temperature data of the user. A baseline is generated for the user using the face coloration video data, the breathing data, and the body temperature data. A health assessment is generated for the user using the baseline, the face coloration video data, the breathing data, and the body temperature data.

### TECHNICAL FIELD

Embodiments described herein generally relate to a personal biometric assessment system, and in an embodiment, but not by way of limitation, a personal biometric assessment system that uses microphones and cameras of personal computer devices.

### BACKGROUND

The vast majority of personal computing devices, whether they are a lap top personal computer, a smartphone, or another type of device, include some types of cameras and microphones. The cameras capture a video of the user or sense the presence of a user, and the microphones capture the voice of the user. However, the use of these data from the cameras and microphones are presently limited to permitting the user of the personal computing device to be seen and heard during online meetings, encounters, and other interactions.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings.

FIG. 1 is an illustration of example breathing patterns.

FIG. 2 is a block diagram of an embodiment of a personal biometric assessment system.

FIGS. 3A, 3B, and 3C are a block diagram illustrating operations and features of a personal biometric assessment system.

FIG. 4 illustrates an example heat map of a face of a user of a personal computing device.

### DETAILED DESCRIPTION

An embodiment of the present disclosure uses the features of a personal computing device to generate a non-medical personal biometric assessment. It relies on specific hardware elements within the personal computing device to make the assessment. These hardware features can include a video camera, a microphone, and an infrared (IR) camera. The embodiment inventories the available hardware to optimize the assessment capabilities of the particular personal computing device. It then uses these features to build an algorithmic assessment for the user. It uses the microphone to record the breathing of the user including amplitude, frequency, content, and rate. It uses a color video camera to assess the color of the user's skin as well as the sheen and pallor. It uses an infrared camera to determine the temperature of the user at various portions of the user's face. In an embodiment, the sensitivity of the microphone can be enhanced to aid in the detection of the breathing, the sensitivity of the video camera can be enhanced to detect the complexion of the user, and the sensitivity of the IR camera can be enhanced to detect small changes in the temperature of the user. Additionally for example, a highly sensitive microphone could also determine the pulse rate of the user. Once this information is collected, the embodiment can assess the health condition of the user. The sensitivity is also used to set the threshold, or base-level for the individual determined algorithmically and using machine learning techniques to titrate rather than a single dimension.

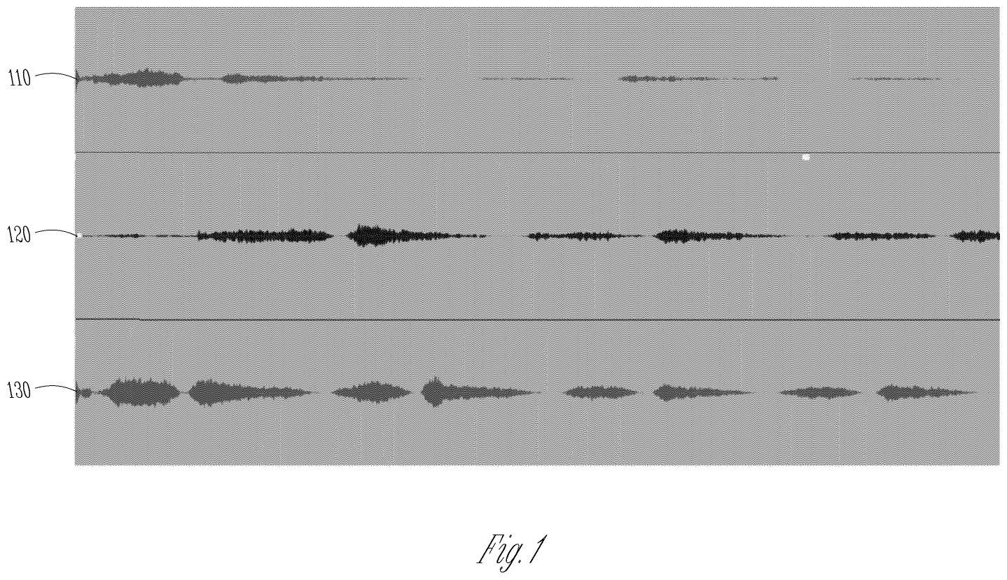

For example, the breathing of the user is assessed using the microphone that is integrated with the personal computing device. FIG. 1 illustrates example breathing patterns of a user. Specifically, a normal pattern is illustrated at 110, a fast pattern is illustrated at 120, and a heavy pattern is illustrated at 130. A pattern of fast breathing 120 and/or heavy breathing 130 can be indicative of stress, and the embodiment can interact with the user to address the situation. For example, if the system detects heavy or fast breathing, it can immediately make suggestions to the user to lower the breathing rate. Then, the system can further assess the breathing to determine if the suggestions had a therapeutic effect. If not, an alarm can be sounded.

As another example, the color of a user's face can be determined with the color video camera that is associated with the personal computing device by looking for hints of specific color indicators, such as red or yellow, to make an assessment. A red facial color could be indicative of stress being experienced by the user.

In yet another example, an infrared (IR) camera can be used to determine the body temperature of the user by examining certain portions of the user's face as discussed in more detail below. Of course, an elevated body temperature could indicate a health issue with the user.

An embodiment provides a unique assessment methodology by establishing a baseline set of characteristics for the user. It then uses machine learning to continuously learn and improve its assessment using feedback from the user. Once the baselining and titration is complete, the system begins making assessments. At this point, the system uses various combinations of breathing, color, and temperature (BCT) to create a BCT pattern that is then used to offer a broad range of guidance to the user.

In a further embodiment, additional devices such as blood pressure monitors, oxygen content monitors (lung and blood), pulse monitors, weight scales, and other devices can be coupled to the personal computing device and data therefrom used in the assessment. In such an embodiment, the system can require the user to opt into or opt out of such additional features because of the privacy issues associated with obtaining additional health data of the user.

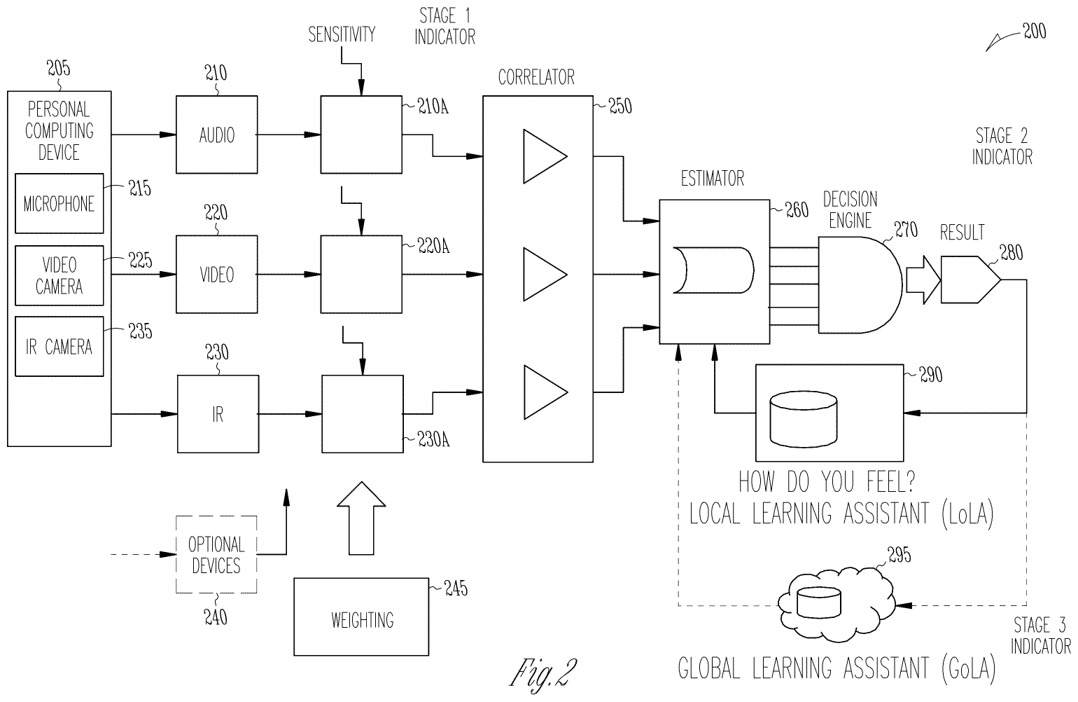

FIG. 2 is a block diagram of an embodiment of a personal biometric assessment system 200. The system 200 receives audio input 210, facial color video input 220, and facial body temperature input 230 from the microphone 215, video camera 225, and infrared camera 235 that are integrated with a personal computing device 205. As illustrated in FIG. 2, other bio-assessment devices 240 such as blood pressure monitors, oxygen content monitors (lung and blood), pulse monitors, and weight scales can be coupled to the system 200. Sensitivity modules 210A, 220A, and 230A receive the audio, video, and infrared data respectively, and determine if there are any data of concern such as heavy/fast breathing, red facial color, and/or elevated body temperature. A weighting module 245 provides weighting factors to the system to identify data that the system considers particularly important. For example, the system may require body temperatures to be at least two degrees above normal and a breathing rate to be at least ten breaths per minute before any concerns are reported to the user. In another embodiment, high frequency notes and/or tones could be sensed in the breathing, and via the machine learning or other means, could inform the system of some aspect of the health of the user. Additionally, if these frequency data or other breathing data indicate a potential health issue, then the system could turn to other aspects of the system such as the facial color data to determine if the facial color data supplements and/or confirms these breathing data.

The audio data 210, the video data 220, and the temperature data 230 are then provided first to a correlator 250, then to an estimator 260, and then to a decision engine or discriminator 270. The correlator 250 conjoins the input signals from the video camera, the microphone, and the infrared camera. If other biometric data are present, such as blood pressure data, those other data are also conjoined. After being conjoined, the data are transmitted to the estimator 260, which searches for signals in the correlator 250 using an artificial intelligence algorithm. The estimator 260 uses both local trend data, external population data and key markers to perform a graduated set of calculations to determine the severity of an indicator. After the estimator 260 has completed its search, the data are transmitted to the discriminator 270, which evaluates signals from the estimator 260 using the artificial intelligence algorithm. The discriminator 270 then uses the various indications of severity to evaluate, against preestablished templates, an establish a set of symptoms that may be contributors to be used in an assessment. The discriminator is also able to establish a time sequence-based algorithm to determine a different outcome even given two similar or identical patterns. By establishing a time sequence t0, t1, t2 . . . as samples it can use the “first indicator” as a primary cause, for example pallor being anemia with slowed breathing being one of many other effects.

The output of the discriminator is a result or health assessment 280. Feedback can be received from the user into a local learning assistance module 290, and this feedback can be provided to the estimator 260 for reassessing the health assessment of the user based on the user's feedback. Additionally, a global learning assistant module 295 can use feedback from a population of users to reassess the health assessment of the user. For example, if the body temperatures of many users in a particular physical space are reported as elevated (perhaps because of a problem with the air conditioning), then this factor can be taken into the user's health assessment.

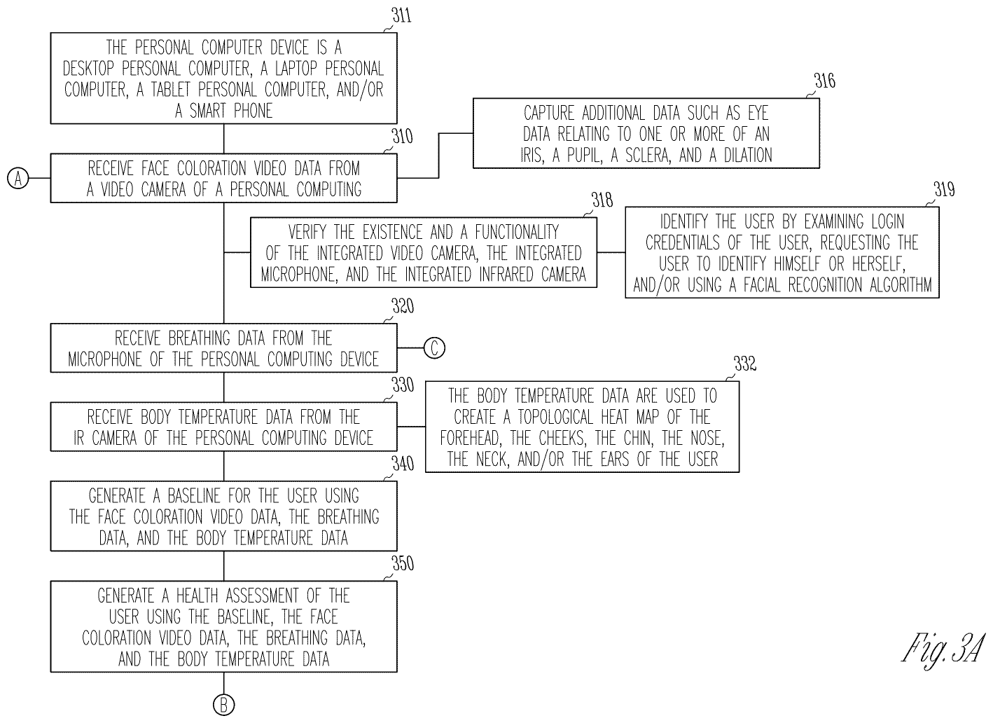

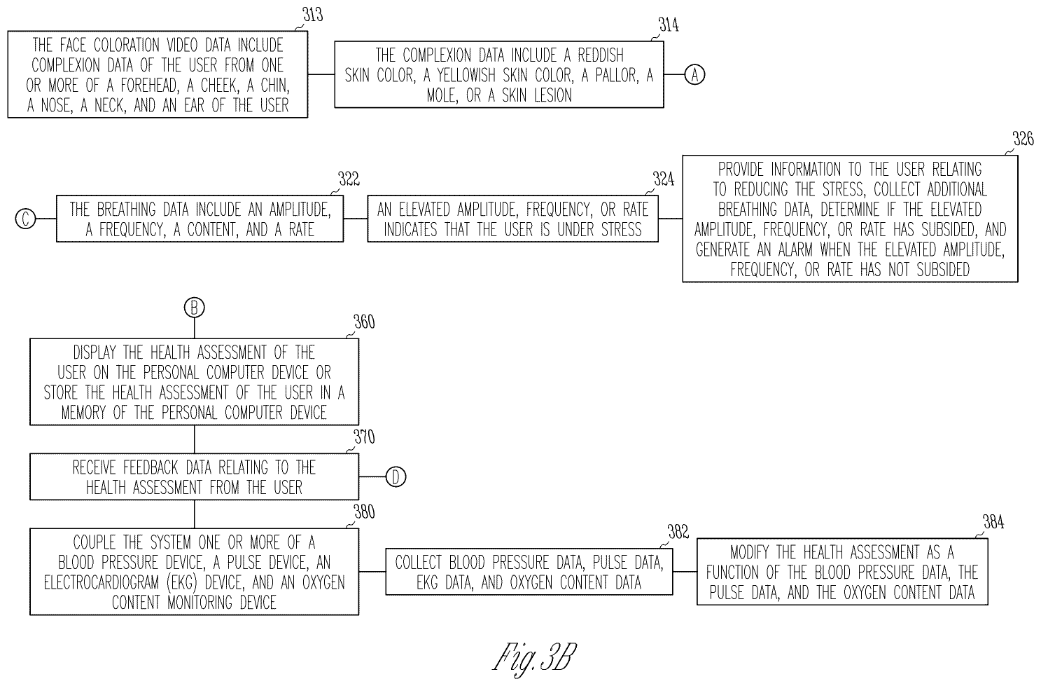

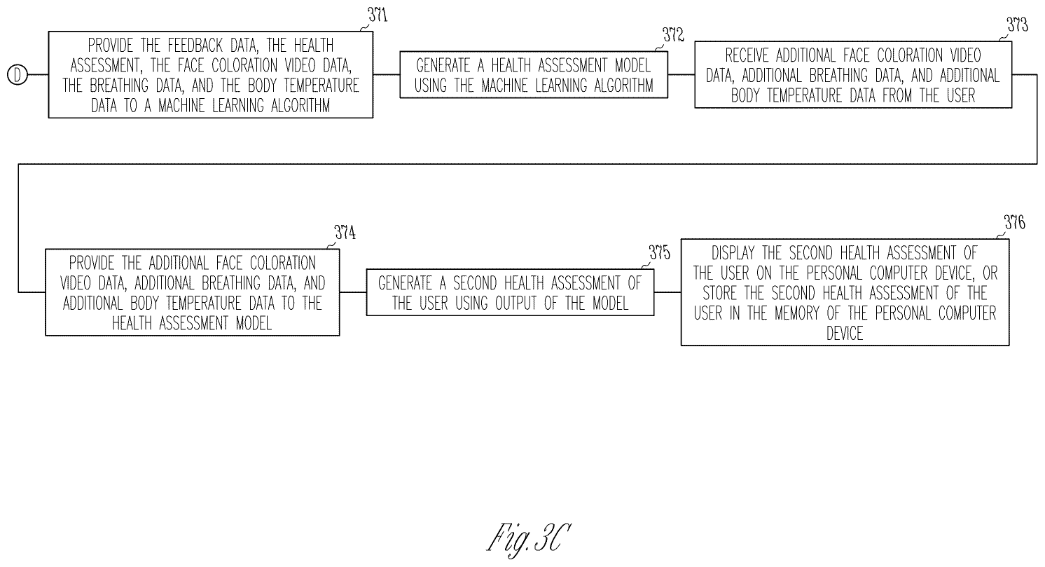

FIGS. 3A, 3B, and 3C are a block diagram illustrating operations and features of a personal biometric assessment system. FIGS. 3A, 3B, and 3C include a number of feature and process blocks 310-384. Though arranged substantially serially in the example of FIGS. 3A, 3B, and 3C, other examples may reorder the blocks, omit one or more blocks, and/or execute two or more blocks in parallel using multiple processors or a single processor organized as two or more virtual machines or sub-processors. Moreover, still other examples can implement the blocks as one or more specific interconnected hardware or integrated circuit modules with related control and data signals communicated between and through the modules. Thus, any process flow is applicable to software, firmware, hardware, and hybrid implementations.

The operations and features of FIGS. 3A, 3B, and 3C can be performed or associated with the system 200 of FIG. 2. Specifically, the personal computing device 205 of FIG. 2 can include a video camera 225 integrated with the personal computer device, a microphone 215 integrated with the personal computer device, and an infrared (IR) camera 235 integrated with the personal computer device.

Now, referring specifically to FIGS. 3A, 3B, and 3C, face coloration video data from the video camera of the personal computing device are received from a user at 310, breathing data from the microphone of the personal computing device are received from the user at 320, and body temperature data from the IR camera of the personal computing device are received from the user at 330. As indicated at 311, the personal computer device is a desktop personal computer, a laptop personal computer, a tablet personal computer, and/or a smart phone. And as indicated at 313, the face coloration video data can include complexion data of the user from one or more of a forehead, a cheek, a chin, a nose, a neck, and an ear of the user. The complexion data can include a reddish skin color, a yellowish skin color, a pallor, a mole, or a skin lesion (314). In an embodiment, if something such as a skin lesion is detected, which may be indicative of a cancer, the user can be instructed to lower and/or tilt his or her head so that the system can record data from the hairline of the user or the top of the user's head. Alternatively, the system, if appropriately equipped, could do a three-dimensional scan of the user's head. At 316, additional data such as eye data relating to one or more of an iris, a pupil, a sclera, and a dilation can be captured. In this embodiment, it may be helpful if there is an enhanced video camera that is associated with the personal computer device. At 318, the computer processor verifies an existence and a functionality of the integrated video camera, the integrated microphone, and the integrated infrared camera. That is, the system first determines the existence of these devices so that the system knows it can begin monitoring for a health assessment. At 319, the system identifies the user by examining login credentials of the user, requesting the user to identify himself or herself, and/or using a facial recognition algorithm. Such user identification is of course necessary for some aspects of the system, such as using the machine learning algorithm to learn about the health of the particular user.

The breathing data can include an amplitude, a frequency, a content, and a rate (322). In particular, if the breathing data include an elevated amplitude, frequency, or rate at 324, this may indicate that the user is under stress, and appropriate actions can be taken. If the data indicate that the user may be under stress, the system can provide information to the user relating to reducing the stress, collect additional breathing data, determine if the elevated amplitude, frequency, or rate has subsided, and generate an alarm when the elevated amplitude, frequency, or rate has not subsided (326).

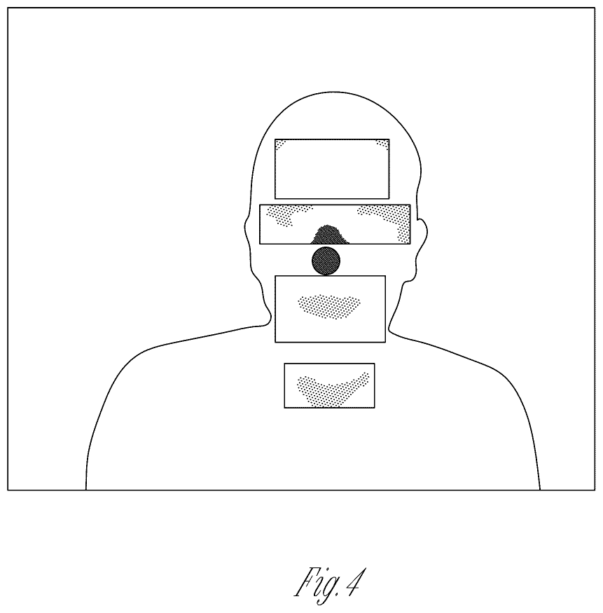

As indicated a 332, the body temperature data can be used to create a topological heat map of the forehead, the cheeks, the chin, the nose, the neck, and/or the ears of the user. An example of such a topological heat map is illustrated in FIG. 4.

At 340, a baseline is generated for the user using the face coloration video data, the breathing data, and the body temperature data. Then, after the generation of the baseline, at 350, a health assessment of the user is generated using the baseline, the face coloration video data, the breathing data, and the body temperature data. At 360, the health assessment of the user is displayed on the personal computer device, or the health assessment of the user is stored in the memory of the personal computer device.

After the health assessment is provided to the user at 360, then at 370, feedback data relating to the health assessment are received from the user. At 371, the feedback data, the health assessment, the face coloration video data, the breathing data, and the body temperature data are provided to a machine learning algorithm. At 372, a health assessment model is generated using the machine learning algorithm. At 373, additional face coloration video data, additional breathing data, and additional body temperature data are received from the user. These additional face coloration video data, additional breathing data, and additional body temperature data are provided to the health assessment model at 374. At 375, a second health assessment of the user is generated using output of the model. At 376, the second health assessment of the user is displayed on the personal computer device, or the second health assessment of the user is stored in the memory of the personal computer device.

As indicated at 380, the system can be coupled to one or more of a blood pressure device, a pulse device, an electrocardiogram (EKG) device, and an oxygen content monitoring device. If the system is so coupled, the system at 382 can collect blood pressure data, pulse data, EKG data, and oxygen content data. Then, at 384, the system can modify the health assessment as a function of the blood pressure data, the pulse data, and the oxygen content data.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

### Examples

Example No. 1 is a system including a personal computer device comprising a computer processor and a memory; a video camera integrated with the personal computer device; a microphone integrated with the personal computer device; and an infrared camera integrated with the personal computer device. The system is operable for receiving face coloration video data of a user from the video camera; receiving breathing data of the user from the microphone; receiving body temperature data of the user from the infrared camera; generating a baseline for the user using the face coloration video data, the breathing data, and the body temperature data; generating a health assessment of the user using the baseline, the face coloration video data, the breathing data, and the body temperature data; and displaying the health assessment of the user on the personal computer device or storing the health assessment of the user in the memory of the personal computer device.
Example No. 2 includes all the features of Example No. 1, and optionally includes a system for receiving feedback data from the user relating to the health assessment; providing the feedback data, the health assessment, the face coloration video data, the breathing data, and the body temperature data to a machine learning algorithm; generating a health assessment model using the machine learning algorithm; receiving additional face coloration video data, additional breathing data, and additional body temperature data from the user; providing the additional face coloration video data, additional breathing data, and additional body temperature data to the health assessment model; generating a second health assessment of the user using output of the model; and displaying the second health assessment of the user on the personal computer device or storing the second health assessment of the user in the memory of the personal computer device.
Example No. 3 includes all the features of Example Nos. 1-2, and optionally includes a system wherein the personal computer device comprises one or more of a desktop personal computer, a laptop personal computer, a tablet personal computer, or a smart phone.
Example No. 4 includes all the features of Example Nos. 1-3, and optionally includes a system wherein the face coloration video data comprise complexion data of the user from one or more of a forehead, a cheek, a chin, a nose, a neck, and an ear of the user.
Example No. 5 includes all the features of Example Nos. 1-4, and optionally includes a system wherein the complexion data comprise one or more of a reddish skin color, a yellowish skin color, a pallor, a mole, or a skin lesion.
Example No. 6 includes all the features of Example Nos. 1-5, and optionally includes a system including eye data relating to one or more of an iris, a pupil, a sclera, and a dilation.
Example No. 7 includes all the features of Example Nos. 1-6, and optionally includes a system wherein the breathing data comprise one of more of an amplitude, a frequency, a content, and a rate.
Example No. 8 includes all the features of Example Nos. 1-7, and optionally includes a system wherein an elevated amplitude, frequency, or rate indicates that the user is under stress.
Example No. 9 includes all the features of Example Nos. 1-8, and optionally includes a system that provides information to the user relating to reducing the stress; collecting additional breathing data; determining if the elevated amplitude, frequency, or rate has subsided; and generating an alarm when the elevated amplitude, frequency, or rate has not subsided.
Example No. 10 includes all the features of Example Nos. 1-9, and optionally includes a system that creates a topological heat map of one or more of a forehead, a cheek, a chin, a nose, a neck, or an ear of the user.
Example No. 11 includes all the features of Example Nos. 1-10, and optionally includes a system that is coupled to one or more of a blood pressure device, a pulse device, an electrocardiogram (EKG) device, and an oxygen content monitoring device; collecting blood pressure data, pulse data, EKG data, and oxygen content data; and modifying the health assessment as a function of the blood pressure data, the pulse data, and the oxygen content data.
Example No. 12 includes all the features of Example Nos. 1-11, and optionally includes a system wherein the computer processor verifies an existence and a functionality of the integrated video camera, the integrated microphone, and the integrated infrared camera.
Example No. 13 includes all the features of Example Nos. 1-12, and optionally includes a system that identifies the user by one or more of examining login credentials of the user, requesting the user to identify themselves, or using a facial recognition algorithm.
Example No. 14 includes all the features of Example Nos. 1-13, and optionally includes a system wherein the computer processor comprises a correlator processor, the correlator processor operable for conjoining three or more input signals from the video camera, the microphone, and the infrared camera.
Example No. 15 includes all the features of Example Nos. 1-14, and optionally includes a system wherein the computer processor comprises an estimator processor coupled to the correlator processor, the estimator processor operable for searching for signals in the correlator processor using an artificial intelligence algorithm.
Example No. 16 includes all the features of Example Nos. 1-15, and optionally includes a system wherein the computer processor comprises a discriminator processor coupled to the estimator processor, the discriminator processor operable for evaluating signals from the estimator processor using the artificial intelligence algorithm.
Example No. 17 is a process including receiving face coloration video data of a user from a video camera integrated with a personal computer device; receiving breathing data of the user from a microphone integrated with the personal computer device; receiving body temperature data of the user from an infrared camera integrated with the personal computer device; generating a baseline for the user using the face coloration video data, the breathing data, and the body temperature data; generating a health assessment of the user using the baseline, the face coloration video data, the breathing data, and the body temperature data; and displaying the health assessment of the user on the personal computer device or storing the health assessment of the user in a memory of the personal computer device.
Example No. 18 includes all the features of Example No. 17, and optionally includes receiving feedback data from the user relating to the health assessment; providing the feedback data, the health assessment, the face coloration video data, the breathing data, and the body temperature data to a machine learning algorithm; generating a health assessment model using the machine learning algorithm; receiving additional face coloration video data, additional breathing data, and additional body temperature data from the user; providing the additional face coloration video data, additional breathing data, and additional body temperature data to the health assessment model; generating a second health assessment of the user using output of the model; and displaying the second health assessment of the user on the personal computer device or storing the second health assessment of the user in the memory of the personal computer device.
Example No. 19 is a machine-readable medium comprising instructions that when executed by a processor executes a process including receiving face coloration video data of a user from a video camera integrated with a personal computer device; receiving breathing data of the user from a microphone integrated with the personal computer device; receiving body temperature data of the user from an infrared camera integrated with the personal computer device; generating a baseline for the user using the face coloration video data, the breathing data, and the body temperature data; generating a health assessment of the user using the baseline, the face coloration video data, the breathing data, and the body temperature data; and displaying the health assessment of the user on the personal computer device or storing the health assessment of the user in a memory of the personal computer device.
Example No. 20 includes all the features of Example No. 19, and optionally includes receiving feedback data from the user relating to the health assessment; providing the feedback data, the health assessment, the face coloration video data, the breathing data, and the body temperature data to a machine learning algorithm; generating a health assessment model using the machine learning algorithm; receiving additional face coloration video data, additional breathing data, and additional body temperature data from the user; providing the additional face coloration video data, additional breathing data, and additional body temperature data to the health assessment model; generating a second health assessment of the user using output of the model; and displaying the second health assessment of the user on the personal computer device or storing the second health assessment of the user in the memory of the personal computer device.

1. A system comprising:
a personal computer device comprising a computer processor and a memory;
a video camera integrated with the personal computer device;
a microphone integrated with the personal computer device; and
an infrared camera integrated with the personal computer device;
wherein the system is operable for:
receiving face coloration video data of a user from the video camera;
receiving breathing data of the user from the microphone;
receiving body temperature data of the user from the infrared camera;
generating a baseline for the user using the face coloration video data, the breathing data, and the body temperature data;
generating a health assessment of the user using the baseline, the face coloration video data, the breathing data, and the body temperature data; and
displaying the health assessment of the user on the personal computer device or storing the health assessment of the user in the memory of the personal computer device.

2. The system of claim 1, comprising:
receiving feedback data from the user relating to the health assessment;
providing the feedback data, the health assessment, the face coloration video data, the breathing data, and the body temperature data to a machine learning algorithm;
generating a health assessment model using the machine learning algorithm;
receiving additional face coloration video data, additional breathing data, and additional body temperature data from the user;
providing the additional face coloration video data, additional breathing data, and additional body temperature data to the health assessment model;
generating a second health assessment of the user using output of the model; and
displaying the second health assessment of the user on the personal computer device or storing the second health assessment of the user in the memory of the personal computer device.

3. The system of claim 1, wherein the personal computer device comprises one or more of a desktop personal computer, a laptop personal computer, a tablet personal computer, or a smart phone.

4. The system of claim 1, wherein the face coloration video data comprise complexion data of the user from one or more of a forehead, a cheek, a chin, a nose, a neck, and an ear of the user.

5. The system of claim 4, wherein the complexion data comprise one or more of a reddish skin color, a yellowish skin color, a pallor, a mole, or a skin lesion.

6. The system of claim 1, comprising eye data relating to one or more of an iris, a pupil, a sclera, and a dilation.

7. The system of claim 1, wherein the breathing data comprise one of more of an amplitude, a frequency, a content, and a rate.

8. The system of claim 7, wherein an elevated amplitude, frequency, or rate indicates that the user is under stress.

9. The system of claim 8, comprising providing information to the user relating to reducing the stress; collecting additional breathing data; determining if the elevated amplitude, frequency, or rate has subsided; and generating an alarm when the elevated amplitude, frequency, or rate has not subsided.

10. The system of claim 1, comprising creating a topological heat map of one or more of a forehead, a cheek, a chin, a nose, a neck, or an ear of the user.

11. The system of claim 1, wherein the system is operable for coupling to one or more of a blood pressure device, a pulse device, an electrocardiogram (EKG) device, and an oxygen content monitoring device; collecting blood pressure data, pulse data, EKG data, and oxygen content data; and modifying the health assessment as a function of the blood pressure data, the pulse data, and the oxygen content data.

12. The system of claim 1, wherein the computer processor verifies an existence and a functionality of the integrated video camera, the integrated microphone, and the integrated infrared camera.

13. The system of claim 1, comprising identifying the user by one or more of examining login credentials of the user, requesting the user to identify themselves, or using a facial recognition algorithm.

14. The system of claim 1, wherein the computer processor comprises a correlator processor, the correlator processor operable for conjoining three or more input signals from the video camera, the microphone, and the infrared camera.

15. The system of claim 14, wherein the computer processor comprises an estimator processor coupled to the correlator processor, the estimator processor operable for searching for signals in the correlator processor using an artificial intelligence algorithm.

16. The system of claim 15, wherein the computer processor comprises a discriminator processor coupled to the estimator processor, the discriminator processor operable for evaluating signals from the estimator processor using the artificial intelligence algorithm.

17. The system of claim 1, wherein the face coloration video data, the breathing data, and the body temperature data are received at a plurality of different times, thereby generating a plurality of sample sets of data and using the plurality of sample sets of data for generating the health assessment.

18. A process comprising:
receiving face coloration video data of a user from a video camera integrated with a personal computer device;
receiving breathing data of the user from a microphone integrated with the personal computer device;
receiving body temperature data of the user from an infrared camera integrated with the personal computer device;
generating a baseline for the user using the face coloration video data, the breathing data, and the body temperature data;
generating a health assessment of the user using the baseline, the face coloration video data, the breathing data, and the body temperature data; and
displaying the health assessment of the user on the personal computer device or storing the health assessment of the user in a memory of the personal computer device.

19. The process of claim 18, comprising:
receiving feedback data from the user relating to the health assessment;
providing the feedback data, the health assessment, the face coloration video data, the breathing data, and the body temperature data to a machine learning algorithm;
generating a health assessment model using the machine learning algorithm;
receiving additional face coloration video data, additional breathing data, and additional body temperature data from the user;
providing the additional face coloration video data, additional breathing data, and additional body temperature data to the health assessment model;
generating a second health assessment of the user using output of the model; and
displaying the second health assessment of the user on the personal computer device or storing the second health assessment of the user in the memory of the personal computer device.

20. A non-transitory machine-readable medium comprising instructions that when executed by a processor executes a process comprising:
receiving face coloration video data of a user from a video camera integrated with a personal computer device;
receiving breathing data of the user from a microphone integrated with the personal computer device;
receiving body temperature data of the user from an infrared camera integrated with the personal computer device;
generating a baseline for the user using the face coloration video data, the breathing data, and the body temperature data;
generating a health assessment of the user using the baseline, the face coloration video data, the breathing data, and the body temperature data; and
displaying the health assessment of the user on the personal computer device or storing the health assessment of the user in a memory of the personal computer device.
